(19) United States
(12) Patent Application Publication
(10) Pub. No.: US 2008/0305048 A1
(43) Pub. Date: Dec. 11, 2008

(54) Self-Assembling Nanoparticle Conjugates

(75) Inventors: Lee Josephson, Reading, MA (US); Ralph Weissleder, Peabody, MA (US); Jesus Manuel Perez, Boston, MA (US)

Correspondence Address: FISH & RICHARDSON PC, P.O. BOX 1022, MINNEAPOLIS, MN, 55440-1022, US

(73) Assignee: THE GENERAL HOSPITAL CORPORATION, Boston, MA (US)

(21) Appl. No.: 10/581,417

(22) PCT Filed: Dec. 9, 2004
(86) PCT No.: PCT/US2004/041300

§ 371 Date: Aug. 14, 2008

Related U.S. Application Data
(60) Provisional application No. 60/528,407, filed on Dec. 10, 2003.

(51) Int. Cl. A61K 49/00; C12Q 1/00; C12Q 1/37; C07D 209/04; C07F 15/02; C12Q 1/26

(52) U.S. Cl. 424/9.32; 435/4; 435/23; 435/28; 556/138; 548/402

(57) ABSTRACT

This invention relates to magnetic nanoparticle conjugates and related compositions and methods of use.

### CROSS REFERENCE TO RELATED APPLICATIONS

This application claims the benefit of U.S. Provisional Application No. 60/528,407, filed on Dec. 10, 2003, the contents of which is incorporated by reference in its entirety.

### STATEMENT REGARDING FEDERALLY SPONSORED RESEARCH

The work described herein was carried out, at least in part, using funds from a federal grant (the Cancer Institute P50 Center Grant (CA86355) and Career Award (CA101781). The government therefore has certain rights in the invention.

### TECHNICAL FIELD

This invention relates to magnetic nanoparticle conjugates and related compositions and methods of use.

### BACKGROUND

Non-invasive imaging of molecular expression in vivo with high resolution and high sensitivity would be a useful tool in clinical diagnostics and in biomedical research. Magnetic resonance imaging (MRI) offers certain well-known advantages as a non-invasive imaging technology. For example, MRI can potentially provide exceptionally high anatomic resolution approaching single-cell levels (voxel of 20-40 μm³). Moreover, recent innovations in instrument design and contrast agent development indicate that even higher resolution can be achieved non-invasively in vivo.

One application of nanotechnology in medicine is the development of biocompatible nanomaterials as environmentally sensitive sensors and molecular imaging agents. Preparations of magnetic particles designed for separation and extraction use particles that are amenable to easy manipulation by weak applied magnetic fields. These materials are typically micron sized and have a high magnetic moment per particle; their effects on water relaxation rate are unspecified and not relevant to their application. Nanoparticles do not respond to the weak, magnetic fields of hand held magnets. Thus, biocompatible nanoparticles with unique optical and/or magnetic properties could have in vitro and in vivo diagnostic applications. The ability to image specific enzyme activities using such nanoparticles would have applications for detecting a variety of diseases and evaluating targeted therapies in individual patients.

### SUMMARY

This invention relates to magnetic nanoparticle conjugates and related compositions and methods of use.

In one aspect this invention relates to compositions having at least two nanoparticle conjugates, each nanoparticle conjugate having a magnetic nanoparticle; and at least one substrate moiety, in which each substrate moiety is linked to the nanoparticle and is chemically modified when the conjugate interacts with a target enzyme. When the target enzyme is absent, the nanoparticle conjugates are essentially monodisperse in liquids; and when the target enzyme is present, the nanoparticle conjugates self-assemble into one or more nanoparticle conjugate clusters through the formation of intermolecular linkages between the chemically modified substrate moieties.

Embodiments can include one or more of the following features.

The conjugates can further include functional groups (e.g., amino, —NHC(O)(CH₂)ₙC(O)—, carboxy, or sulfhydryl groups, in which n is 0-100, e.g., n can be 6) that link the nanoparticle to one or more substrate moieties.

The magnetic nanoparticles each can include a magnetic metal oxide (e.g., a superparamagnetic metal oxide). The metal oxide can be iron oxide. In some embodiments, the nanoparticles can be amino-derivatized cross-linked iron oxide nanoparticles.

The substrate moieties can include a phenolic moiety, and can be chemically modified by oxidation (e.g., one electron oxidation).

The target enzyme can be a protease or a peroxidase (e.g., a myeloperoxidase or horseradish peroxidase).

Each of the monodisperse nanoparticle conjugates can have an average particle size of between about 40 nm and about 60 nm. In some embodiments, each of the monodisperse nanoparticle conjugates can have an average particle size of about 50 nm.

Each of the nanoparticle conjugate clusters can have an average particle size of between about 400 nm and about 500 nm. In some embodiments, each of the nanoparticle conjugate clusters can have an average particle size of about 450 nm.

Each of the monodisperse nanoparticle conjugates can have an R1 relaxivity between about 5 and 30 mM¹ sec⁻¹ and an R2 relaxivity between about 15 and 100 mM⁻¹ sec⁻¹.

The intermolecular linkages can be covalent linkages or non-covalent linkages.

The formation of intermolecular linkages between the chemically modified substrate moieties can be irreversible.

The formation of intermolecular linkages between the chemically modified substrate moieties can result in crosslinking of the nanoparticle conjugates.

The composition can further include a fluid media.

Self-assembly of the nanoparticle conjugates can result in the spin-spin relaxation time of the fluid being decreased relative to the spin-spin relaxation time of the fluid having essentially only monodisperse nanoparticle conjugates present. The decrease in spin-spin relaxation time can be dependent upon the concentration of the target enzyme.

The nanoparticle conjugate can have a formula X-(L)x-A, in which X is a magnetic nanoparticle; L is —NH—, —NHC(O)(CH₂)ₙC(O)—, —C(O)O—, or —SS—, in which n is 0-20; A is substituted or unsubstituted aryl, substituted or unsubstituted heteroaryl, substituted or unsubstituted aralkyl, substituted or unsubstituted heteroaralkyl, substituted or unsubstituted aralkylamino, or substituted or unsubstituted heteroaralkylamino; wherein substitutents are selected from halo, hydroxy, C₁-C₄ alkoxy, or C₁-C₄ alkyl; and x is 0 or 1. X can be magnetic metal oxide (e.g., iron oxide). x can be 1 and L can be —NHC(O)(CH₂)ₙC(O)— (e.g., n can be 6). A can be substituted aralkylamino, or substituted heteroaralkylamino. In some embodiments, A is substituted with at least one hydroxyl group, and A can be

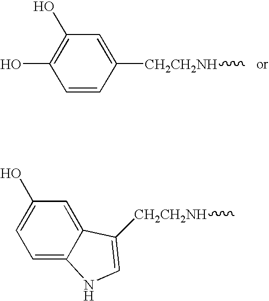

In some embodiments, the composition can include a population of at least two nanoparticle conjugates, in which at least one nanoparticle conjugate has a magnetic nanoparticle and/or substrate moiety that is different from the magnetic nanoparticle and/or substrate moiety of one or more members in the population. For example, a population can include one or more first nanoparticle conjugates, each including a first magnetic nanoparticle and a first substrate moiety, and one or more second nanoparticle conjugates, each including a second magnetic nanoparticle and a second substrate moiety, whereby two types of nanoparticle conjugates are present. The first and second magnetic nanoparticles can be different and/or the first and second substrate moieties can be different. The compositions can include a plurality of different types of conjugates (e.g., 3, 4, 5, 6, 7, 8, 9, 10, 50, 90, 96, 100, 150, 200, 250, 300, 350, 360, 364, 400, or 500 types).

In another aspect, this invention relates to in vitro methods for detecting the presence of a target enzyme in a sample, the method includes (i) providing a composition including at least two of the new nanoparticle conjugates described herein; (ii) contacting the composition with a fluid sample; (iii) allowing time (a) for the target enzyme to contact the nanoparticle conjugates and (b) for the nanoparticle conjugates to self-assemble into clusters through the formation of intermolecular linkages between the chemically modified substrate moieties; and (iv) determining the spin-spin relaxation time of the fluid over time. A decrease in spin-spin relaxation time indicates the presence of the target enzyme in the sample.

In some embodiments, the methods further include the addition of hydrogen peroxide or glucose oxidase.

In a further aspect, this invention relates to in vivo methods for detecting the presence of a target enzyme in a subject (e.g., a human) by (i) administering to the subject a composition including at least two of the new nanoparticle conjugates described herein; (ii) allowing time (a) for the target enzyme to contact the nanoparticle conjugates and (b) for the nanoparticle conjugates to self-assemble into clusters through the formation of intermolecular linkages between the chemically modified substrate moieties; and (iii) determining the spin-spin relaxation time of the fluid over time. A decrease in spin-spin relaxation time indicates the presence of the target enzyme in the subject.

The methods can further include the step of identifying the subject as being in need of such detection.

In one aspect, this invention relates to the new self-assembling, nanoparticle conjugates having a magnetic nanoparticle; and at least one substrate moiety, in which each substrate moiety is linked to the nanoparticle and is chemically modified when the conjugate interacts with a target enzyme. When two or more nanoparticle conjugates are present and when the target enzyme is absent, the nanoparticle conjugates are essentially monodisperse in a liquid; and when two or more nanoparticle conjugates are present and when the target enzyme is present, the nanoparticle conjugates self-assemble into one or more nanoparticle conjugate clusters through the formation of intermolecular linkages between the chemically modified substrate moieties.

In some embodiments, the conjugates can have a formula X-(L)x-A, in which in which X is a magnetic nanoparticle; L is —NH—, —NHC(O)—, —NHC(O)(CH₂)ₙC(O)—, —C(O)O—, or —SS—, in which n is 0-20; A is substituted or unsubstituted aryl, substituted or unsubstituted heteroaryl, substituted or unsubstituted aralkyl, substituted or unsubstituted heteroaralkyl, substituted or unsubstituted aralkylamino, or substituted or unsubstituted heteroaralkylamino; wherein substitutents are selected from halo, hydroxy, C₁-C₄ alkoxy, or C₁-C₄ alkyl; and x is 0 or 1. X can be magnetic metal oxide (e.g., iron oxide). x can be 1 and L can be —NHC(O)(CH₂)ₙC(O)— (e.g., n can be 6). A can be substituted aralkylamino, or substituted heteroaralkylamino. In some embodiments, A is substituted with at least one hydroxyl group, and A can be

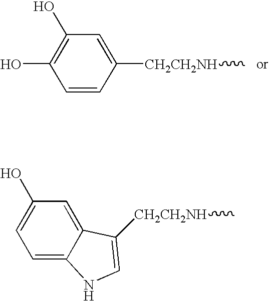

In another aspect, this invention relates to a packaged product including a composition having at least two of the new nanoparticle conjugates described herein.

Embodiments may include one or more of the following advantages.

In all embodiments, the nanoparticle conjugates are essentially monodispersed in the absence of a target enzyme, which can reduce the likelihood that the conjugates are cleared by the reticuloendothelial system prior to interaction with a target enzyme. Thus, the conjugates have relatively long circulation times in vivo.

In all embodiments, a single particle preparation is administered for imaging, which reduces the likelihood of observing multiple, differing pharmacokinetic profiles that can sometimes be associated with multi-particle preparations.

In some embodiments, the nanoparticle conjugates contain phenolic moieties as substrate moieties, in which relatively straightforward substitutions of the aromatic ring can result in incremental changes in the redox properties of the aromatic ring, thus allowing the substrate moieties to be readily tuned to different enzyme selectivities. Thus, a variety of target enzyme specific conjugates can be readily designed and prepared from the same basic nanoparticle scaffold.

In some embodiments, a single enzyme can result in the self-assembly of a plurality of nanoparticle conjugates, thereby achieving biological amplification at relatively low nanoparticle conjugate concentrations.

In some embodiments, preferential changes in R2 relaxivity can allow R1 relaxivity/R2 relaxivity magnetic resonance imaging to provide data that can be useful for measuring target enzyme concentrations.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and from the claims.

### DESCRIPTION OF DRAWINGS

FIG. 1A is a graphical representation of the particle size distribution by light scattering of the dopamine nanoparticle conjugates before incubation with horse radish peroxidase (HRP).

FIG. 1B is a graphical representation of the particle size distribution by light scattering of the dopamine nanoparticle conjugates after incubation with HRP.

FIG. 2 is a graphical representation of the effects of increasing HRP concentration on the δT2 of a solution containing dopamine nanoparticle conjugates with (solid squares) and without (solid triangles) hydrogen peroxide.

FIG. 3 is a graphical representation of the effects of increasing the amount of sodium azide (inhibitor) on the δT2 of a solution containing dopamine nanoparticle conjugates with hydrogen peroxide.

FIG. 4A is a graphical representation of δT2 values of the serotonin nanoparticle conjugates in the presence of increasing amounts of myeloperoxidase detected using a 1.5T clinical MRI both with (solid squares) and without (solid triangles) hydrogen peroxide.

FIG. 4B is a magnetic resonance image (1.5T MRI) corresponding to the following myeloperoxidase activity data point shown in FIG. 4A: with peroxide; 0.0031 units/μL MPO.

FIG. 4C is a magnetic resonance image (1.5T MRI) corresponding to the following myeloperoxidase activity data point shown in FIG. 4A: with peroxide; 0.0061 units/μL MPO.

FIG. 4D is a magnetic resonance image (1.5T MRI) corresponding to the following myeloperoxidase activity data point shown in FIG. 4A: with peroxide; 0.0125 units/μL MPO.

FIG. 4E is a magnetic resonance image (1.5T MRI) corresponding to the following myeloperoxidase activity data point shown in FIG. 4A: with peroxide; 0.025 units/μL MPO.

FIG. 4F is a magnetic resonance image (1.5T MRI) corresponding to the following myeloperoxidase activity data point shown in FIG. 4A: without peroxide; 0.0031 units/μL MPO.

FIG. 4G is a magnetic resonance image (1.5T MRI) corresponding to the following myeloperoxidase activity data point shown in FIG. 4A: without peroxide; 0.0061 units/μL MPO.

FIG. 4H is a magnetic resonance image (1.5T MRI) corresponding to the following myeloperoxidase activity data point shown in FIG. 4A: without peroxide; 0.0125 units/μL MPO.

FIG. 4I is a magnetic resonance image (1.5T MRI) corresponding to the following myeloperoxidase activity data point shown in FIG. 4A: without peroxide; 0.025 units/μL MPO.

FIG. 4J is a T2 (msec) magnetic resonance image signal intensity level scale corresponding to the magnetic resonance images shown in FIGS. 4B-4I. The levels shown in FIGS. 4B-4E occur in the top half of the scale, and the levels of FIGS. 4F-4I occur in the bottom half of the scale. The level shown in FIG. 4B occurs at about the top of the scale.

FIG. 5A is a magnetic resonance image (1.5T MRI) of myeloperoxidase activity (0 units/mL MPO) using dopamine-nanoparticle conjugates. There was essentially no difference in signal intensity observed between this image and the images shown in FIGS. 5B, 5C, and 5D.

FIG. 5B is a magnetic resonance image (1.5T MRI) of myeloperoxidase activity (0.0061 units/μL MPO) using dopamine-nanoparticle conjugates. There was essentially no difference in signal intensity observed between this image and the images shown in FIGS. 5A, 5C, and 5D.

FIG. 5C is a magnetic resonance image (1.5T MRI) of myeloperoxidase activity (0.025 units/μL MPO) using dopamine-nanoparticle conjugates. There was essentially no difference in signal intensity observed between this image and the images shown in FIGS. 5A, 5B, and 5D.

FIG. 5D is a magnetic resonance image (1.5T MRI) of myeloperoxidase activity (0 units/μL MPO) using serotonin-nanoparticle conjugates. There was essentially no difference in signal intensity observed between this image and the images shown in FIGS. 5A, 5B, and 5C.

FIG. 5E is a magnetic resonance image (1.5T MRI) of myeloperoxidase activity (0.0061 units/μL MPO) using serotonin-nanoparticle conjugates.

FIG. 5F is a magnetic resonance image (1.5T MRI) of myeloperoxidase activity (0.025 units/μL MPO) using serotonin-nanoparticle conjugates.

FIG. 5G is a T2 (msec) magnetic resonance image signal intensity level scale corresponding to the magnetic resonance images shown in FIGS. 5A-5F. The levels shown in FIGS. 5A-5D occur at about the top of the scale, the level of FIG. 5E occurs in the top half of the scale. The level shown in FIG. 5F occurs at about the bottom of the scale.

Like reference symbols in the various drawings indicate like elements.

### DETAILED DESCRIPTION

### General

This invention relates to magnetic nanoparticle conjugates and related compositions and methods of use. The nanoparticle conjugates generally include a magnetic nanoparticle (circled “NP” in Scheme 1 below), that is linked to at least one substrate moiety (circled “S” in Scheme 1 below). The nanoparticle conjugates may optionally contain functional groups that link one or more substrate moieties to the nanoparticle. The substrate moiety can be any chemical group that can participate in an enzyme (e.g., a target enzyme)-mediated chemical reaction. As such, one or more nanoparticle-bound substrate moieties can be chemically modified (shaded circled “S” in Scheme 1 below) upon interaction of the conjugates with the target enzyme (e.g., a peroxidase, a protease). When the target enzyme interacts with a population of two or more nanoparticle conjugates, the conjugates can self-assemble into nanoparticle conjugate clusters through the formation of intermolecular (i.e., interconjugate) linkages between the chemically modified substrate moieties. In the absence of a target enzyme, the nanoparticle conjugates are essentially monodispersed (e.g., in solution or in a nonhomogenous fluid media).

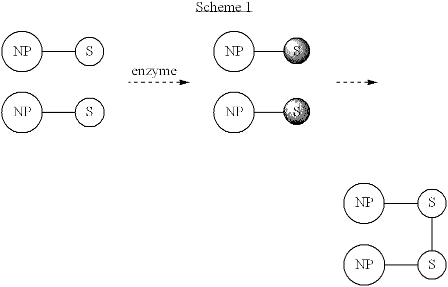

In general, the clusters formed from the nanoparticle conjugates described herein have one or more measurable properties (e.g., magnetic properties), that are altered, (e.g., increased or decreased) relative to the same one or more measurable properties of the monodispersed nanoparticle conjugates. For example, the solvent (e.g., water) spin-spin relaxation times (T2) for solution phase nanoparticle conjugate clusters are relatively low in magnitude and differentiable, (e.g., by nuclear magnetic resonance (NMR) or magnetic resonance imaging (MRI)), from the relatively high solvent spin-spin relaxation times for the corresponding monodispersed, solution phase nanoparticle conjugates. Accordingly, it is believed that solvent spin-spin relaxation times can be a useful parameter for determining the presence or absence of a target enzyme in biological samples containing nanoparticle conjugates with target enzyme-specific substrate moities. While not wishing to be bound by theory, it is believed that magnetic resonance amplification in the form of a decrease in T2 would be observed in samples containing the target enzyme because interaction of the monodispersed nanoparticle conjugates (high T2) with the target enzyme results in the formation of one or more clusters (low T2), thereby decreasing the observed T2 of the sample.

### DEFINITIONS

The term “alkyl” refers to a hydrocarbon chain that may be a straight chain or branched chain, containing the indicated number of carbon atoms. For example, C₁˜C₁₂ alkyl indicates that the group may have from 1 to 12 (inclusive) carbon atoms in it. The term “aralkyl” refers to an alkyl moiety in which one or more alkyl hydrogen atoms is replaced by an aryl group. Examples of “aralkyl” include benzyl, 2-phenylethyl, 3-phenylpropyl, 9-fluorenyl, benzhydryl, and trityl groups. The term “heteroaralkyl” refers to an alkyl moiety in which one or more alkyl hydrogen atoms is replaced by an heteroaryl group. Examples of “heteroaralkyl” include, e.g., tryptaminyl.

The terms “aralkylamino” and “diaralkylamino” refer to —NH(aralkyl) and —N(aralkyl)₂ radicals respectively. The terms “heteroaralkylamino” and “diheteroaralkylamino” refer to —NH(heteroaralkyl) and —N(heteroaralkyl)₂ radicals respectively The term “alkoxy” refers to an —O-alkyl radical.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution can be substituted by a substituent.

The term “substituents” refers to a group “substituted” on an alkyl, cycloalkyl, alkenyl, alkynyl, heterocyclyl, heterocycloalkenyl, cycloalkenyl, aryl, aralkyl, heteroaralkyl, heteroaryl, aralkylamino, diaralkylamino, heteroaralkylamino, or diheteroaralkylamino group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

The term “halo” or “halogen” refers to any radical of fluorine, chlorine, bromine or iodine.

The term “alkylene” refers to a divalent alkyl, e.g., —CH₂—, —CH₂CH₂—, and —CH₂CH₂CH₂—.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The term “cycloalkyl” as employed herein includes saturated cyclic, bicyclic, tricyclic, or polycyclic hydrocarbon groups having 3 to 12 carbons. Any ring atom can be substituted. The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted. The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted. The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted. The heterocycloalkenyl groups can contain fused rings. Fused rings are rings that share a common carbon atom. Examples of heterocycloalkenyl include but are not limited to tetrahydropyridyl and dihydropyranyl.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The terms “aminocarbonyl,” alkoxycarbonyl,” hydrazinocarbonyl, and hydroxyaminocarbonyl refer to the radicals —C(O)NH₂, —C(O)O(alkyl), —C(O)NH₂NH₂, and —C(O)NH₂NH₂, respectively.

The term “interacts” refers to any contact, reaction, or binding that occurs between a nanoparticle conjugate and a target enzyme.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as being one or more of the predominant resonance forms for a particular species.

### Structure of Nanoparticle Conjugates

In all embodiments the nanoparticle component of the conjugate is a magnetic nanoparticle, (e.g., magnetic metal oxide, such as superparamagnetic iron oxide). The magnetic metal oxide can also comprise cobalt, magnesium, zinc, or mixtures of these metals with iron. The term “magnetic” as used herein means materials of high positive magnetic susceptibility such as superparamagnetic compounds and magnetite, gamma ferric oxide, or metallic iron. Preferred nanoparticles include those having a relatively high relaxivity, i.e., strong effect on water relaxation.

In all embodiments, at least one substrate moiety is covalently linked to the nanoparticle. In some embodiments, the substrate moiety is linked to the nanoparticle via a functional group. The functional group can be chosen or designed primarily on factors such as convenience of synthesis, lack of steric hindrance, and biodegradation properties. Suitable functional groups may include —NH—, —NHNH—, —O—, —S—, —SS—, —C(O)O—, —C(O)S—, —NHC(O)(CH₂)ₙC(O)—, —NHC(O)—, —OC(O)(CH₂)ₙ(O)—, —OC(O)(CH₂)ₙC(O)—, —C(O)(CH₂)ₙC(O)—, —NH(CH₂)ₙC(O)—, —O(CH₂)ₙC(O)—, —S(CH₂)ₙC(O)—, —NH(CH₂)ₙ—, —O(CH₂)ₙ—, or —S(CH₂)ₙ—, in which n is 1-100 (e.g., x is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 96, 97, 98, 99). Functional groups having cyclic, unsaturated, or cyclic unsaturated groups in place of the linear and fully saturated alkylene linker portion, (CH₂)ₙ, may also be used to attach substrate moieties to the nanoparticle. In some embodiments, the functional group is —NHC(O)(CH₂)₆C(O)—. The functional group may be present on a starting material or synthetic intermediate that is associated with either the nanoparticle or the substrate moiety.

The number of substrate moieties linked to a nanoparticle may be selected as desired. In some embodiments, a nanoparticle starting material can contain one or more functional groups for attachment of substrate moieties, (e.g., 2, 4, 6, 8, 10, 15, 20, 25, 30, 35, 40, 45, or 50 functional groups). The number of substrate moieties that are ultimately linked to the nanoparticle can either be equal to or less than the number of functional groups that are available for attachment to the nanoparticle. In some embodiments, the number of substrate moieties linked can correspond to a number that may be necessary to maintain monodispersion of the conjugates in the absence of the target enzyme. In some embodiments, the steric bulk of the substrate moiety or the nature of the enzyme being targeted can also be determinative of the number of substrate moieties that are ultimately loaded on to the nanoparticle. In any event, it is permissible for the number of substrate moieties per nanoparticle conjugate to vary within a given population of two or more nanoparticle conjugates.

The substrate moiety can generally be any chemical group that (1) can function as a substrate for an enzyme (e.g., a target enzyme)-mediated (e.g., catalyzed) chemical reaction; and (2), when chemically modified, can form an intermolecular linkage (e.g., a covalent or noncovalent linkage) with a second, chemically modified substrate moiety. The substrate moiety can be a relatively highly reactive substrate for the target enzyme, which readily undergoes chemical modification upon interaction of the conjugate with the target enzyme. In some embodiments, the substrate moiety is a substrate for a protease or a peroxidase-mediated chemical reaction. In some embodiments, the target enzyme-mediated reaction results in oxidation of the substrate moiety (e.g., a one electron oxidation), to provide a radical as the chemically modified substrate moiety.

In some embodiments, the substrate moiety is a phenolic moiety. As used herein, “phenolic moiety” means a moiety containing a phenolic ring. As used herein, a “phenolic ring” is a phenyl ring wherein at least one ring position is substituted with a hydroxyl (OH) group, and other ring positions are optionally substituted, provided that at least one ring position is unsubstituted (see structures A and B below). In some embodiments, the phenyl ring may further contain a fused heteroaryl ring (e.g., structure B).

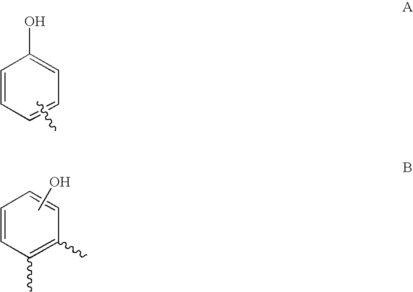

Numerous structural variations are permissible in the phenolic moiety, and the phenolic moiety can be substituted with electron donating or withdrawing groups so as to alter the electronic properties (e.g., the redox properties), of the aromatic ring π-electron system. For example, the ortho and/or para positions relative to the hydroxyl group can be substituted with OH, or C₁-C₄ alkoxy (e.g., OCH₃). When both para positions are substituted, the substituents can be the same or different. In another variation, an amino group or an amido group is substituted at a meta position on the phenolic ring. The effect(s) of the various substitutions possible on the phenolic ring can be predicted by one of skill in the art according to known principles of organic chemistry, based on the identities of the substituents and their relative positions on the ring. See, e.g., L. G. Wade, Jr., 1988, Organic Chemistry, Prentice-Hall, Inc., Englewood Cliffs, N.J. at 666-669. For example, an amino group at the meta position (relative to the hydroxyl group) is relatively strongly activating, i.e., this substitution enhances the electron donor ability of the aromatic ring.

Under certain conditions, phenolic moieties can function as electron donors in enzyme-catalyzed reductions (e.g., a peroxidase-catalyzed reduction of hydrogen peroxide). Oxidation, (e.g., one electron oxidation), of a phenolic moiety can provide a free radical intermediate, (e.g., a tyrosyl radical), which, in turn, may couple with a second free radical intermediate, (e.g., a second tyrosyl radical), to form a covalent carbon-carbon single bond between the two radical intermediates (see Scheme 2 below). Carbon-carbon bond formation may occur in an intermolecular manner, resulting in, for example, cross-linking of the two phenolic moieties. One electron reduction of phenolic moieties and cross linking of tyrosyl radicals are described in the art, (e.g., Heinecke, J. W. Free Radic Biol Med 2002, 32, 1090-1011; Heinecke, J. W., et al. J Biol Chem 1993, 268, 4069-4077; Winterbourn, C. C., et al. Biochem Biophys Res Commun 2003, 305, 729-736; McConnick, M. L., et al. J Biol Chem 1998, 273, 32030-32037).

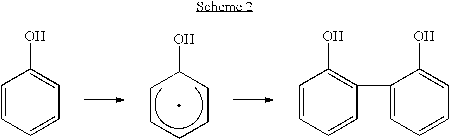

Accordingly, nanoparticle conjugates having phenolic substrate moities (Structure I in Scheme 3 below) can be useful for detecting the presence of target enzymes that mediate reductions, (e.g., peroxidases). While not wishing to be bound by theory, it is hypothesized that interaction of I with such a target enzyme would provide structure II (see Scheme 3 below) in which the substrate moieties have been chemically modified to form free radicals via one electron oxidation. The enzyme-induced formation of these radicals would then be followed by result in subsequent intermolecular, ortho, ortho cross-linking between the chemically modified phenolic substrate moieties to provide the self-assembly III, (see Scheme 3 below), providing measurable changes in the magnetic resonance signal.

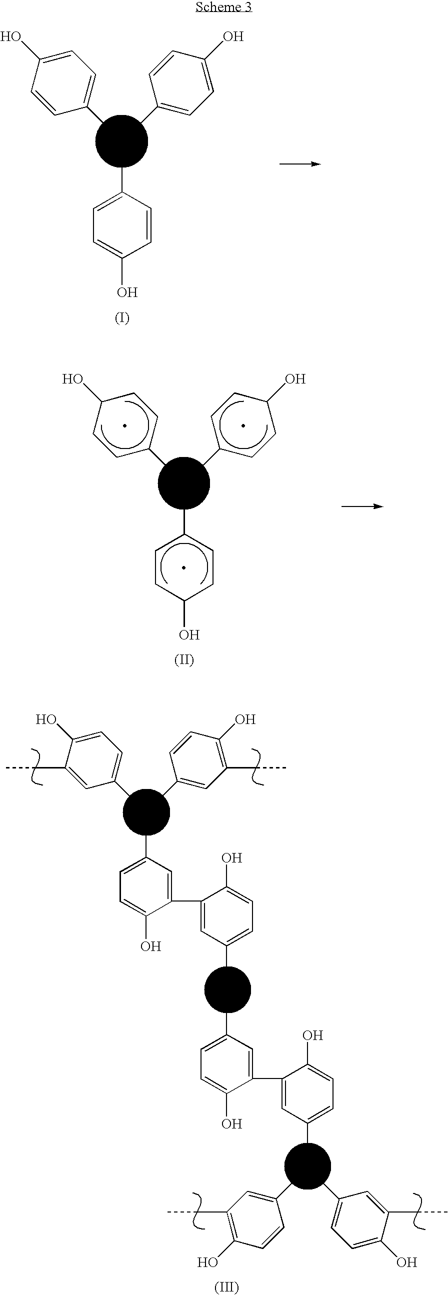

One subset of nanoparticle conjugates has a formula X-(L)x-A, in which:

X is a magnetic nanoparticle;

L is a functional group that may include —NH—, —NHC(O)—, —NHC(O)(CH₂)ₙC(O)—, —C(O)O—, or —SS—, in which n is 0-20;

A is a substrate moiety that may include substituted or unsubstituted aryl, substituted or unsubstituted heteroaryl, substituted or unsubstituted aralkyl, substituted or unsubstituted heteroaralkyl, substituted or unsubstituted aralkylamino, or substituted or unsubstituted heteroaralkylamino; wherein substitutents are selected from amino, halo, hydroxy, C₁-C₄ alkoxy, or C₁-C₄ alkyl; and x is 0 or 1.

A useful subset includes those conjugates in which X is an iron oxide nanoparticle, x is 1, L is —NHC(O)(CH₂)₆C(O)—, and A is aralkylamino substituted with at least one hydroxyl group, (e.g., Structure C in which the substrate moiety is derived from dopamine) or heteroaralkylamino substituted with at least one hydroxyl group (e.g., Structure D in which the substrate moiety is derived from serotonin).

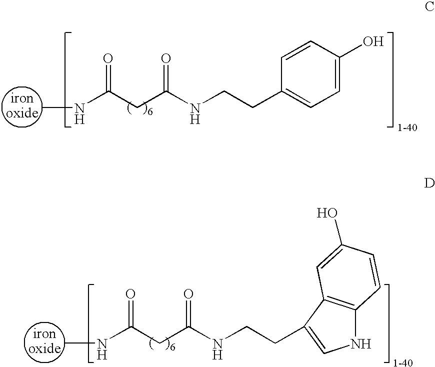

In general, the overall size of the nanoparticle conjugates is about 15 to 200 nm, e.g., about 20 to 100 nm, about 40 to 60 nm; or about 50 nm. The metal oxides are crystals of about 1-25 nm, e.g., about 3-10 nm, or about 5 nm in diameter.

The conjugates have a relatively high relaxivity owing to the superparamagnetism of their iron or metal oxide. They have an R1 relaxivity between about 5 and 30 mM⁻¹ sec⁻¹, e.g., 10, 15, 20, or 25 mM⁻¹ sec⁻¹. They have an R2 relaxivity between about 15 and 100 mM⁻¹ sec⁻¹, e.g., 25, 50, 75, or 90 mM⁻¹ sec⁻¹. They typically have a ratio of R2 to R1 of between 1.5 and 4, e.g., 2, 2.5, or 3. They typically have an iron oxide content that is greater than about 10% of the total mass of the particle, e.g., greater than 15, 20, 25 or 30 percent.

### Synthesis of Nanoparticle Conjugates

In some embodiments, nanoparticles having functional groups, (e.g., electrophilic functional groups such as carboxy groups or nucleophilic groups such as amino groups) can be employed as starting materials for the nanoparticle conjugates.

Carboxy functionalized nanoparticles can be made, for example, according to the method of Gorman (see WO 00/61191). In this method, reduced carboxymethyl (CM) dextran is synthesized from commercial dextran. The CM-dextran and iron salts are mixed together and are then neutralized with ammonium hydroxide. The resulting carboxy functionalized nanoparticles can be used for coupling amino functionalized groups, (e.g., a further segment of the functional group or the substrate moiety).

Carboxy-functionalized nanoparticles can also be made from polysaccharide coated nanoparticles by reaction with bromo or chloroacetic acid in strong base to attach carboxyl groups. In addition, carboxy-functionalized particles can be made from amino-functionalized nanoparticles by converting amino to carboxy groups by the use of reagents such as succinic anhydride or maleic anhydride.

Nanoparticle size can be controlled by adjusting reaction conditions, for example, by using low temperature during the neutralization of iron salts with a base as described in U.S. Pat. No. 5,262,176. Uniform particle size materials can also be made by fractionating the particles using centrifugation, ultrafiltration, or gel filtration, as described, for example in U.S. Pat. No. 5,492,814.

Nanoparticles can also be synthesized according to the method of Molday (Molday, R. S. and D. MacKenzie, “Immunospecific ferromagnetic iron-dextran reagents for the labeling and magnetic separation of cells,” J. Immunol. Methods, 1982, 52(3):353-67, and treated with periodate to form aldehyde groups. The aldehyde-containing nanoparticles can then be reacted with a diamine (e.g., ethylene diamine or hexanediamine), which will form a Schiff base, followed by reduction with sodium borohydride or sodium cyanoborohydride.

Dextran-coated nanoparticles can be made and cross-linked with epichlorohydrin. The addition of ammonia will react with epoxy groups to generate amine groups, see Hogemann, D., et al., Improvement of MRI probes to allow efficient detection of gene expression Bioconjug. Chem. 2000. 11(6):941-6, and Josephson et al., “High-efficiency intracellular magnetic labeling with novel superparamagnetic-Tat peptide conjugates,” Bioconjug. Chem., 1999, 10(2):186-91. This material is known as cross-linked iron oxide or “CLIO” and when functionalized with amine is referred to as amine-CLIO or NH₂—CLIO.

Carboxy-functionalized nanoparticles can be converted to amino-functionalized magnetic particles by the use of water-soluble carbodiimides and diamines such as ethylene diamine or hexane diamine.

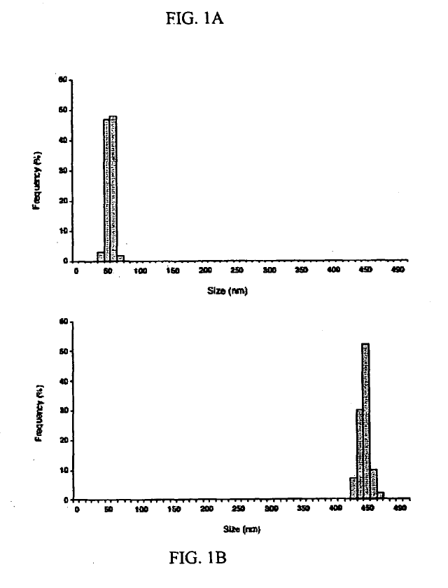

Compounds having structures corresponding to C and D were prepared using amino functionalized dextran-caged superparamagnetic iron oxide nanoparticles were used as the starting material. Dopamine or serotonin was conjugated to the aminated magnetic nanoparticles using suberic acid bis(N-hydroxysuccinimide ester) (DSS, Pierce Co). On average, each nanoparticle starting material had about 40 reactive amino groups, which were used for conjugation. Serotonin attachment was verified through its fluorescent emission at 345 nm. These nanoparticle conjugates were monodispersed in solution, having a narrow particle size distribution as determined by light scattering with an average particles size of about 50 nm. Particle size distribution for the dopamine-containing nanoparticle conjugates is shown in FIG. 1A. The water protons' spin-lattice relaxation (R1) of the nanoparticle conjugates was 25.8 s⁻¹mM⁻¹ while the spin-spin relaxation (R2) was 67 s⁻¹mM⁻¹. Relaxivity and size by light scattering can be determined by the methods described in, for example, Shen, T., et al. Magn. Reson. Med. 29, 599-604.

### Uses of the Nanoparticle Conjugates

Solvent, (e.g., water), spin-spin relaxation times (T2) can be determined by relaxation measurements using a nuclear magnetic resonance benchtop relaxometer. In general, T2 relaxation time measurements can be carried out at 0.47 T and 40° C. (Bruker NMR Minispec, Billerica, Mass.) using solutions with a total iron content of 10 μg Fe/mL.

Alternatively, T2 relaxation times can be determined by magnetic resonance imaging of 384-well plates (50 μL sample volume), allowing parallel measurements at higher throughput. In general, magnetic resonance imaging can be carried out using a 1.5 T superconducting magnet (Sigma 5.0; GE medical Systems, Milwaukee, Wis.) using T2-weighted spin echo sequences with variable echo times (TE=25-1000 ms) and repetition times (TR) of 3,000 ms to cover the spectrum of the anticipated T2 values. This technique is described in, for example, Perez, J. M., et al. Nat Biotechnol 2002, 20, 816-820; and Hogemann, D., et al. Bioconjug Chem 2002, 13, 116-121.

In some embodiments, the magnetic nanoparticle conjugates self-assemble in solution by the action of a specific peroxidase, with the enzyme-mediated magnetic nanoparticle self-assembly acting as a magnetic resonance signal amplification system, which is sensitive to the enzymatic activity of the peroxidase. For an initial proof of the concept, we used horseradish peroxidase (HRP) an enzyme generally used in bioassays, while as a clinically relevant target, we used myeloperoxidase (MPO), an enzyme implicated in atherosclerosis and inflammation (see, for example, Zhang, R., et al, Jama 2001, 286, 2136-2142; Brennan, M. L., et al. N Engl J Med 2003, 349, 1595-1604).

In the aforementioned experiments, dopamine and serotonin were selected and used as substrate moieties in two separates sets of nanoparticle conjugates (e.g., C and D) for detection of HRP and MPO, respectively. These phenolic agents were thus chosen to be electron donors for the peroxidase-catalyzed reduction of hydrogen peroxide.

Generally, when a peroxidase is used in vitro, the new in vitro methods of the invention include providing a suitable amount of hydrogen peroxide in the tissue to be imaged. The hydrogen peroxide can be supplied directly. Alternatively, it can be generated in situ, e.g., using glucose oxidase. If the hydrogen peroxide is enzymatically generated in situ, the generating enzyme can be administered directly (as a pre-formed enzyme) or can be expressed in the tissue from a suitable nucleic acid vector introduced into the tissue.

To test whether incubation of the nanoparticle conjugates with the corresponding peroxidase would result in cluster formation, the dopamine-nanoparticle conjugates (10 μg Fe/mL, 0.1M phosphate pH 6.0) were incubated with HRP (0.9 units/μL) for 2 hours. After this incubation period, cluster formation was readily detectable by light scattering. The particle size distribution for the clusters are shown in FIG. 1B (particle size distribution before incubation are shown in FIG. 1A). As expected, no cluster formation occurred in the absence of H₂O₂. These nanoclusters were stable in aqueous solution, did not continue growing in size and did not precipitate. Similar results were observed when serotonin-nanoparticles were incubated with myeloperoxidase.

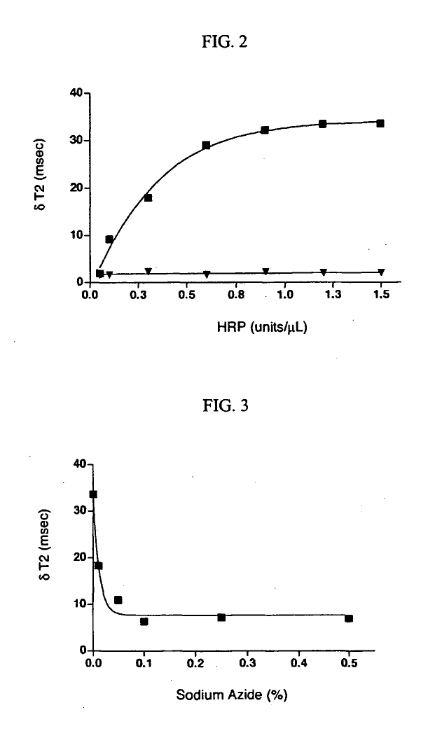

Next, we investigated whether the peroxidase-mediated clustering would result in T2 relaxation time changes (δT2) of the solution. For these experiments, a solution of the HRP targeting nanoparticle conjugate (10 μg Fe/mL, 0.1M phosphate pH 6.0) was incubated with different amounts of HRP (0-0.9 Units/μL) for 2 hours at 4° C. and the T2 relaxation times were measured at 0.47 T. Increasing δT2 values were observed upon incubation with increasing amount of HRP, reaching saturation at a concentration of 0.9 units/mL in this specific experiment as shown in FIG. 2. Essentially no changes in T2 were observed in samples incubated with HRP in the absence of H₂O₂. To further confirm that the detectable changes in T2 are caused by an HRP-mediated mechanism, experiments were performed in which an increasing amount of sodium azide, a known inhibitor of peroxidase, was added to the solution. As expected, sodium azide inhibited HRP activity and reduced the δT2 changes in a concentration dependent manner as shown in FIG. 3. T2 changes did not occur in other control experiments using heat- or SDS-denatured HRP. The above results confirm that the observed changes in δT2 were HRP-specific and that these nanoparticle conjugates can be used as nanosensors for peroxidase activity detection.

The ability of the nanoparticle conjugates to image myeloperoxidase (MPO) activity was tested using a 1.5T clinical MRI imaging system. Recent studies have demonstrated the importance of MPO in the development of inflammation and cardiovascular diseases such as atherosclerosis and myocardial infarction. High levels of intracellular MPO content has been found in plasma samples from patients with coronary heart disease and acute coronary syndromes while many other studies implicate MPO as one of the pathways for the oxidation of low density lipoprotein in the artery wall (see, for example, Heinecke, J. W. Curr Opin Lipidol 1997, 8, 268-274; Savenkova, M. L., et al. J Biol Chem 1994, 269, 20394-20400; Leeuwenburgh, C., et al. J Biol Chem 1997, 272, 3520-3526). It has also been observed that an increased number of MPO-expressing macrophages can occur in eroded or ruptured plaques causing acute coronary syndromes (see, for example, Sugiyama, S.; Okada, Y.; Sukhova, G. K.; Virmani, R.; Heinecke, J. W.; Libby, P. Am J Pathol 2001, 158, 879-89 l).

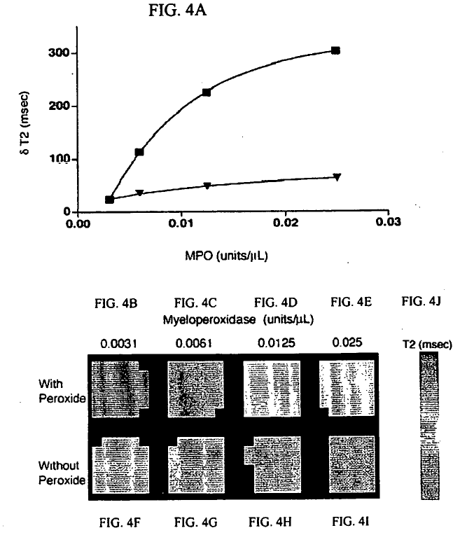

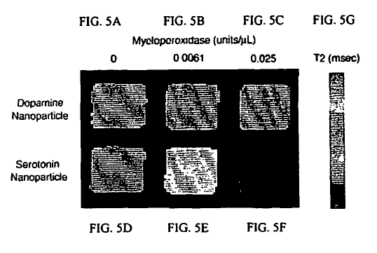

A serotonin-containing nanoparticle conjugate (prepared as described herein) was selected for the MPO imaging experiments because serotonin has been reported to be a superior substrate for myeloperoxidase relative to dopamine (see, for example, Allegra, M., et al. Biochem Biophys Res Commun 2001, 282, 380-386; Dunford, H. B.; Hsuanyu, Y. Biochem Cell Biol 1999, 77, 449-457). The serotonin-nanoparticles (3 μg Fe/mL, 0.1M phosphate pH 6.0) were incubated with various amounts of myeloperoxidase both with and without H₂O₂ in a 384 well-plate and imaged by MRI. Similar to the experiments conducted with HRP, δT2 increased as a function of MPO concentration as shown in FIG. 4A. Furthermore, we were able to demonstrate that δT2 changes were of significant magnitude to be detectable using a clinical MR imaging system (see FIGS. 4B-4E and 4J). Control samples consisting of MPO-nanosensor incubated with myeloperoxidase in the absence of H₂O₂ showed no significant increase in δT2 as expected (see FIGS. 4F-4I and 4J). Likewise, as shown in FIGS. 5A-5G, the dopamine-containing nanoparticle conjugates did not show any δT2 in the presence of MPO (i.e., essentially no difference in signal intensity observed when dopamine-nanoparticle conjugates are incubated with myeloperoxidase). The findings demonstrate that the selectivity of the particle-bound substrate moieties is about the same as that for the particle free substrates.

Substrate moieties are not limited to chemical groups that are substrates for enzyme-mediated oxidation-reduction reactions. Many enzymes known in the art, (e.g., polymerases), catalyze the formation of chemical bonds via different reaction mechanisms.

In magnetic resonance (MR) imaging applications, the nanoparticle conjugates can be used in methods for the detection and a spatial localization of target enzymes in living systems. This is based, in part, on the ability of the magnetic conjugates to effect water relaxation in media that generally will not permit assays using light-based methods. Hence, the conjugates can function as MR contrast agents or magnetic nanosensors for the detection of target enzymes in vivo.

The new conjugates are essentially nontoxic to mammalian cells. The nanoparticle conjugates can be administered to a subject, e.g., a human or animal, such as a mammal (e.g., dogs, cats, cows, pigs, and horses). Various routes of administration known in the art can be used to achieve systemic or local delivery (e.g., orally, parenterally, by inhalation spray, topically, rectally, nasally, buccally, vaginally or via an implanted reservoir). Compositions containing the nanoparticle conjugates of this invention may contain any conventional non-toxic pharmaceutically-acceptable carriers, adjuvants or vehicles (e.g., a fluid media).

Also within the scope of this invention is a method of screening substrate moieties for selectivity for one or more target enzymes. For example, libraries of phenolic substrates attached to nanoparticles can be screened by high throughput NMR methods described herein (e.g., for numerous peroxidases).

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising at least two nanoparticle conjugates, each nanoparticle conjugate comprising:
a magnetic nanoparticle; and
at least one substrate moiety, in which each substrate moiety is linked to the nanoparticle and is chemically modified when the conjugate interacts with a target enzyme; wherein,
when the target enzyme is absent, the nanoparticle conjugates are essentially monodisperse in a liquid; and
when the target enzyme is present, the nanoparticle conjugates self-assemble into one or more nanoparticle conjugate clusters through the formation of intermolecular linkages between the chemically modified substrate moieties.

2. The composition of claim 1, wherein the conjugates further comprise functional groups that link the nanoparticle to one or more substrate moieties.

3. The composition of claim 2, wherein the functional groups are selected from amino, —NHC(O)(CH₂)ₙC(O)—, carboxy, or sulfhydryl groups, wherein n is 0-100.

4. The composition of claim 1, wherein the magnetic nanoparticles each comprise a magnetic metal oxide.

5. The composition of claim 4, wherein the magnetic metal oxide is a superparamagnetic metal oxide.

6. The composition of claim 4, wherein the metal oxide is iron oxide.

7. The composition of claim 4, wherein the nanoparticles are an amino-derivatized cross-linked iron oxide nanoparticles.

8. The composition of claim 1, wherein the substrate moieties comprise a phenolic moiety.

9. The composition of claim 1, wherein the substrate moieties are chemically modified by oxidation.

10. The composition of claim 9, wherein the oxidation is a one electron oxidation.

11. The composition of claim 1, wherein the target enzyme is a protease.

12. The composition of claim 1, wherein the target enzyme is a peroxidase.

13. The composition of claim 12, wherein the peroxidase is myeloperoxidase.

14. The composition of claim 12, wherein the peroxidase is horseradish peroxidase.

15. The composition of claim 1, wherein each of the monodisperse nanoparticle conjugates has an average particle size of between about 40 nm and about 60 nm.

16. The composition of claim 1, wherein each of the monodisperse nanoparticle conjugates has an average particle size of about 50 nm.

17. The composition of claim 1, wherein each of the nanoparticle conjugate clusters has an average particle size of between about 400 nm and about 500 nm.

18. The composition of claim 1, wherein each of the nanoparticle conjugate clusters has an average particle size of about 450 nm.

19. The composition of claim 14, wherein each of the monodisperse nanoparticle conjugates has an R1 relaxivity between about 5 and 30 mM⁻¹ sec⁻¹ and an R2 relaxivity between about 15 and 100 mM⁵¹ sec⁻¹.

20. The composition of claim 1, wherein the intermolecular linkages are covalent linkages.

21. The composition of claim 1, wherein the intermolecular linkages are non-covalent linkages.

22. The composition of claim 1, wherein the formation of intermolecular linkages between the chemically modified substrate moieties is irreversible.

23. The composition of claim 1, wherein the formation of intermolecular linkages between the chemically modified substrate moieties results in crosslinking of the nanoparticle conjugates.

24. The composition of claim 1, wherein the composition further comprises a fluid media.

25. The composition of claim 24, wherein self-assembly of the nanoparticle conjugates results in the spin-spin relaxation time of the fluid being decreased relative to the spin-spin relaxation time of the fluid having essentially only monodisperse nanoparticle conjugates present.

26. The composition of claim 24, wherein the decrease in spin-spin relaxation time is dependent upon the concentration of the target enzyme.

27. The composition of claim 1, wherein the nanoparticle conjugate has a formula
X-(L)x-A, wherein:
X is a magnetic nanoparticle;
L is —NH—, —NHC(O)(CH₂)ₙC(O)—, —C(O)O—, or —SS—, wherein n is 0-20;
A is substituted or unsubstituted aryl, substituted or unsubstituted heteroaryl, substituted or unsubstituted aralkyl, substituted or unsubstituted heteroaralkyl, substituted or unsubstituted aralkylamino, or substituted or unsubstituted heteroaralkylamino; wherein substitutents are selected from halo, hydroxy, C₁-C₄ alkoxy, or C₁-C₄ alkyl; and
x is 0 or 1.

28. The composition of claim 27, wherein X is magnetic metal oxide.

29. The composition of claim 28, wherein the metal oxide is iron oxide.

30. The composition of claim 27, wherein x is 1 and L is —NHC(O)(CH₂)ₙC(O)—.

31. The composition of claim 30, wherein n is 6.

32. The composition of claim 27, wherein A is substituted aralkylamino, or substituted heteroaralkylamino.

33. The composition of claim 32, wherein A is substituted with at least one hydroxyl group.

34. The composition of claim 33, wherein A is:

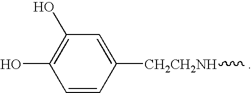

35. The composition of claim 33, wherein A is:

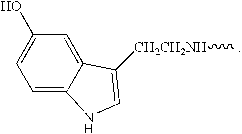

36. An in vitro method for detecting the presence of a target enzyme in a sample, the method comprising:
(i) providing a composition comprising at least two nanoparticle conjugates, each nanoparticle conjugate comprising a magnetic nanoparticle; and at least one substrate moiety, in which each substrate moiety is linked to the nanoparticle and is chemically modified when the conjugate interacts with a target enzyme; wherein, when the target enzyme is absent, the nanoparticle conjugates are essentially monodisperse; and when the target enzyme is present, the nanoparticle conjugates self-assemble into one or more nanoparticle conjugate clusters through the formation of intermolecular linkages between the chemically modified substrate moieties;
(ii) contacting the composition with a fluid sample;
(iii) allowing time (a) for the target enzyme to contact the nanoparticle conjugates and (b) for the nanoparticle conjugates to self-assemble into clusters through the formation of intermolecular linkages between the chemically modified substrate moieties; and
(iv) determining the spin-spin relaxation time of the fluid over time,
wherein a decrease in spin-spin relaxation time indicates the presence of the target enzyme in the sample.

37. The method of claim 37, further comprising the addition of hydrogen peroxide.

38. The method of claim 36, further comprising the addition of glucose oxidase.

39. An in vivo method for detecting the presence of a target enzyme in a subject, the method comprising:
(i) administering to the subject a composition comprising at least two nanoparticle conjugates, each nanoparticle conjugate comprising a magnetic nanoparticle; and at least one substrate moiety, in which each substrate moiety is linked to the nanoparticle and is chemically modified when the conjugate interacts with a target enzyme; wherein, when the target enzyme is absent, the nanoparticle conjugates are essentially monodisperse; and when the target enzyme is present, the nanoparticle conjugates self-assemble into one or more nanoparticle conjugate clusters through the formation of intermolecular linkages between the chemically modified substrate moieties;
(ii) allowing time (a) for the target enzyme to contact the nanoparticle conjugates and (b) for the nanoparticle conjugates to self-assemble into clusters through the formation of intermolecular linkages between the chemically modified substrate moieties; and
(iii) determining the spin-spin relaxation time of the fluid over time,
wherein a decrease in spin-spin relaxation time indicates the presence of the target enzyme in the subject.

40. The method of claim 39, wherein the subject is a human.

41. The method of claim 39, further comprising the step of identifying the subject as being in need of such detection.

42. A self-assembling, nanoparticle conjugate comprising:
a magnetic nanoparticle; and
at least one substrate moiety, in which each substrate moiety is linked to the nanoparticle and is chemically modified when the conjugate interacts with a target enzyme; wherein,
when two or more nanoparticle conjugates are present and when the target enzyme is absent, the nanoparticle conjugates are essentially monodisperse in a liquid; and
when two or more nanoparticle conjugates are present and when the target enzyme is present, the nanoparticle conjugates self-assemble into one or more nanoparticle conjugate clusters through the formation of intermolecular linkages between the chemically modified substrate moieties.

43. The nanoparticle conjugate of claim 42, wherein the conjugate has a formula X-(L)x-A,
wherein:
X is a magnetic nanoparticle;
L is —NH—, —NHC(O)(CH₂)ₙC(O)—, —C(O)O—, or —SS—, wherein n is 0-20;
A is substituted or unsubstituted aryl, substituted or unsubstituted heteroaryl, substituted or unsubstituted aralkyl, substituted or unsubstituted heteroaralkyl, substituted or unsubstituted aralkylamino, or substituted or unsubstituted heteroaralkylamino; wherein substitutents are selected from halo, hydroxy, C₁-C₄ alkoxy, or C₁-C₄ alkyl; and
x is 0 or 1.

44. The conjugate of claim 43, wherein X is magnetic metal oxide.

45. The conjugate of claim 44, wherein the metal oxide is iron oxide.

46. The conjugate of claim 43, wherein x is 1 and L is —NHC(O)(CH₂)ₙC(O)—.

47. The conjugate of claim 46, wherein n is 6.

48. The conjugate of claim 43, wherein A is substituted aralkylamino, or substituted heteroaralkylamino.

49. The conjugate of claim 48, wherein A is substituted with at least one hydroxyl group.

50. The composition of claim 49, wherein A is:

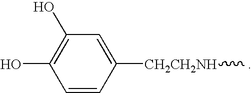

51. The conjugate of claim 49, wherein A is:

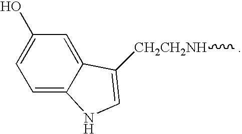

52. A packaged product comprising:
a composition comprising at least two nanoparticle conjugates, each nanoparticle conjugate comprising:
a magnetic nanoparticle; and
at least one substrate moiety, in which each substrate moiety is linked to the nanoparticle and is chemically modified when the conjugate interacts with a target enzyme; wherein,
when the target enzyme is absent, the nanoparticle conjugates are essentially monodisperse in a liquid; and
when the target enzyme is present, the nanoparticle conjugates self-assemble into one or more nanoparticle conjugate clusters through the formation of intermolecular linkages between the chemically modified substrate moieties.
